(19) United States
(12) Patent Application Publication
(10) Pub. No.: US 2007/0213705 A1
(43) Pub. Date: Sep. 13, 2007

(54) INSULATED NEEDLE AND SYSTEM

(76) Inventor: Peter M. Schmid, Longmont, CO (US)

Correspondence Address: THE OLLILA LAW GROUP LLC, 2060 BROADWAY, SUITE 300, BOULDER, CO, 80302, US

(21) Appl. No.: 11/683,262

(22) Filed: Mar. 7, 2007

Related U.S. Application Data
(60) Provisional application No. 60/780,139, filed on Mar. 8, 2006.

(51) Int. Cl. A61B 18/18

(52) U.S. Cl. 606/44

(57) ABSTRACT

The present invention relates to a needle for directly emitting energy to dermal or sub-dermal tissue in a patient. In the presently preferred embodiment, the needle includes an elongated shaft that is provided with a pointed end, a first shaft section, and a second shaft section. The first and second shaft sections include a conductive material. The second shaft section includes an insulated shaft section that is provided with a length dimensioned according to a depth of epidermal tissue.

This application claims the benefit of U.S. Provisional Application No. 60/780,139, filed Mar. 18, 2006. The disclosure of U.S. Provisional Application No. 60/780,139 is hereby incorporated herein by reference.

### FIELD OF THE INVENTION

The present invention relates to a device utilizing energy to reduce the appearance of aging signs in skin tissue.

### BACKGROUND OF THE INVENTION

Currently, there are a variety of procedures in use for reducing the appearance of the aging signs in skin. Some of the common procedures employ muscle paralytics, injection fillers, microdermabrasion and dermabrasion, chemical peels, resurfacing and regeneration systems, or the application of direct energy.

Common muscle paralytics are Botox®, Myobloc®, and Dysport®. These chemodenervating materials are injected through the skin at the subcutaneous level into select muscle groups and act to temporary paralyze select muscle groups, which create dynamic facial wrinkles. The temporary paralysis occurs as a result of blockage at the neural junction affecting the neurotransmitter to the muscle. This sort of treatment has a temporary effect and does not improve photo damaged skin, skin laxity, passive skin wrinkles, or the deep layers of skin.

Injection fillers, such as, Collagen (Cosmoderm®), hyaluronic acid (Restylane®, Juviderm™, Hylaform®, and Captique®), calcium hydroxyapatite (Radiesse®) and others, are selectively injected at various depths in or beneath the skin to “fill” the wrinkle and reduce the surface effect. They, in effect, plump up the skin to reduce the size of the wrinkle. The beneficial effects of these fillers are temporary, or, in the case of silicone or Artefill®, permanent. However, permanent fillers may lead to long term adverse side effects in humans.

In a microdermabrasion or dermabrasion process, the skin is abraded layer by layer to a selected depth. The abrasion promotes injury and healing of the tissue. The healing results in a somewhat favorable deposition of collagen into the dermis and the reduction of wrinkles. However, microdermabrasion is too superficial to cause a significant deep tissue effect and deep dermabrasion is invasive, resulting in side effects, which often lead to permanent unnatural lightening of the skin.

When chemical peeling is employed, acidic chemicals of various strengths and types are applied directly to the skin. Depending on the type of chemical utilized, the acids penetrate the skin to a varying degree. The penetration of the acid promotes injury and favorable healing of the tissue. However, light superficial chemical peels have limited beneficial effects on the skin and deep peels are invasive and result in side effects, which often lead to permanent unnatural lightening of the skin.

Resurfacing and regenerating systems employ energy in the form of heat, which is applied to the skin to create a deep healing response in the dermis. These systems use various technologies, such as, carbon dioxide, erbium, Nd-Yag (neodymium-doped yttrium aluminium garnet), nitrogen plasma gas, or the scanning, pulsing, or the application of fractionated energy to the skin to create a favorable response. These treatments heat the skin causing collagen shrinkage and deep tissue insult, resulting in a healing effect and the deposition of collagen into the dermis; thus, improving the surface appearance of skin. The superficial fractionated systems have limited beneficial aesthetic effects and the deep treatment lasers result in significant downtime, and often lead to permanent lightening of the skin.

The latest treatments of the signs of aging utilize direct energy, such as, light emitted diodes, ultrasound energy, and monopolar and bipolar radiofrequency, having unique and specific properties. During treatment, energy is applied to the skin by noninvasive hand pieces. The energy emitted by the hand pieces transmits through the top surface layer of the skin to promote a favorable response. Nonablative monopolar radiofrequency (MRF) treatment (ThermaCool™, Thermage Inc., Hayward, Calif.) is one nonablative rejuvenation modality. However, since these treatments are noninvasive, they have very limited beneficial aesthetic results.

The present invention is directed to a device configured to directly apply energy to specific sub-surface levels of the skin, to promote a favorable healing effect.

### SUMMARY OF THE INVENTION

The scope of the present invention is defined solely by the appended claims, and is not affected to any degree by the statements within this summary. Briefly stated, the invention relates to a needle for directly emitting energy to dermal or sub-dermal tissue in a patient. In the presently preferred embodiment, the needle include an elongated shaft that is provided with a pointed end, a first shaft section, and a second shaft section. The first and second shaft sections include a conductive material. The second shaft section includes an insulated shaft section that is provided with an insulated shaft section length that is dimensioned according to a depth of epidermal tissue.

### BRIEF DESCRIPTION OF THE DRAWINGS

FIG. 1 depicts a perspective view of a needle of an embodiment of the present invention connected to a wire connected to an adaptor.

FIG. 2 depicts a perspective view of an angulated needle of an embodiment of the present invention.

FIG. 3 depicts a perspective view of a shortened needle of an embodiment of the present invention.

FIG. 4 depicts a perspective view of a narrow diameter needle of an embodiment of the present invention.

FIG. 5 depicts an embodiment of a needle delivery system including a pad sheathing a plurality of conductive wires connected to a plurality of needles.

FIG. 6 is a side view depicting the embodiment of a needle delivery system shown in FIG. 5.

FIG. 7 is a close up side view depicting the embodiment of a needle delivery system shown in FIG. 5 in relation to epidermal and dermal tissue.

### DETAILED DESCRIPTION OF THE PRESENTLY PREFERRED EMBODIMENTS

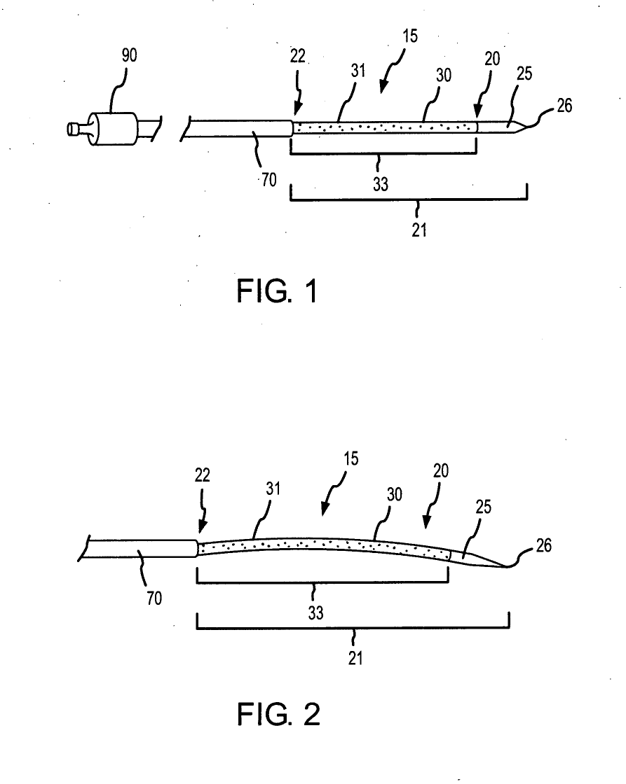

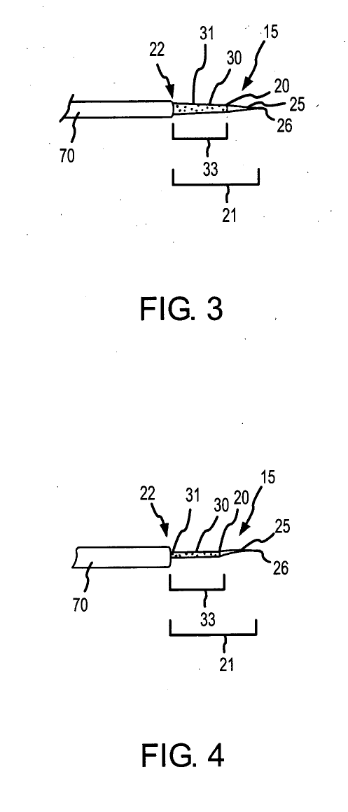

FIGS. 1-4 depict needles 15 according to embodiments of the present invention. As shown, the needles 15 are provided with an elongated shaft 20 that includes a length 21. According to one aspect of the presently preferred embodiment the length 21 of the shaft 20 is dimensioned according to the depth 51 of epidermal tissue 50 in a typical patient. As shown in FIG. 7, in the presently preferred embodiment, the length 21 is dimensioned to be greater than the depth 51 of the epidermal tissue 50 in a patient.

As shown in FIGS. 1-4, the elongated shaft 20 includes a first shaft section 25. According to one aspect of the presently preferred embodiment, the first shaft section 25 is configured to deliver energy to dermal tissue 60 in a patient. According to another aspect of the presently preferred embodiment, the first shaft section 25 is configured to deliver energy to sub-dermal tissue (not shown) in a patient. According to yet another aspect of the presently preferred embodiment, the first shaft section 25 is configured to pierce the epidermal tissue 50 in a patient, whereby the first shaft section 25 can be inserted into the dermal tissue 60 of a patient. According to still another aspect of the presently preferred embodiment, the first shaft section 25 is configured to pierce the epidermal tissue 50 in a patient, whereby the first shaft section 25 can be inserted into the sub-dermal tissue (not shown) of a patient.

In the presently preferred embodiment, the first shaft section 25 is fabricated from a conductive material, such as, a metal, for example, a stainless steel. As shown in FIGS. 1-4, the first shaft section 25 preferably extends from a pointed end 26 of the shaft 20. Turning now to FIG. 7, the pointed end 26 permits the shaft 20 to penetrate the epidermal tissue 50 of a patient, whereby the first shaft section 25 can be inserted into the dermal tissue 60 or sub-dermal tissue (not shown) for energy treatment.

As shown in FIGS. 1-4, the elongated shaft 20 includes a second shaft section 30. According to one aspect of the presently preferred embodiment, the second shaft section 30 is configured to protect epidermal tissue 50 of a patient from energy damage. According to another aspect of the presently preferred embodiment, the second shaft section 30 is configured to conduct energy to the first shaft section 25. According to yet another aspect of the presently preferred embodiment, the second shaft section 30 contacts a conductive wire 70. According to still another aspect of the presently preferred embodiment, the second shaft section 30 is connected to a conductive wire 70.

In the presently preferred embodiment, the second shaft section 3 0 is fabricated to include a conductive material, such as, a metal, for example, a stainless steel. As shown in FIGS. 1-4 the second shaft section 30 includes all insulating section 31, which is preferably applied as a coating of insulating material, such as, for example, Teflon®. As shown in FIGS. 1-4, the insulating section 31 extends along the second shaft section 30 for a predetermined length 33, According to one aspect of the presently preferred embodiment, the length 33 is dimensioned according to the depth 51 of epidermal tissue 50 in a typical patient. As shown in FIG. 7, in the presently preferred embodiment, tie length 33 is dimensioned to be greater than or equal to the depth 51 of the epidermal tissue 50 in a patient.

In the presently preferred embodiment, at least a portion of second shaft section 30 is provided with a contact portion 32 (shown in FIG. 7) of conductive material. In the presently preferred embodiment, the contact portion 32 is positioned to contact the wire 70, whereby it is capable of conducting energy from the wire 70, which, in turn, is conducted by the second shaft section 30 to the first shaft section 25.

In the embodiments depicted, the contact portion 32 is a generally flat surface shown preferably located on the outer end 21 of the shaft 20; however, it is within the scope of the present invention to provide the contact portion 32 with any number of shapes. By way of example, and not limitation, the contact portion 32 can define a socket and/or include an axially extending section or sections provided with an outer surface or outer surfaces including any number of diameters and/or shapes. Moreover, although in the presently preferred embodiment, the wire 70 directly contacts the contact portion 32 to conduct the energy to the second shaft section 30, it is within the scope of the present invention to provide any number of intervening conductive structures which conduct the energy from the wire to the second shaft section 30.

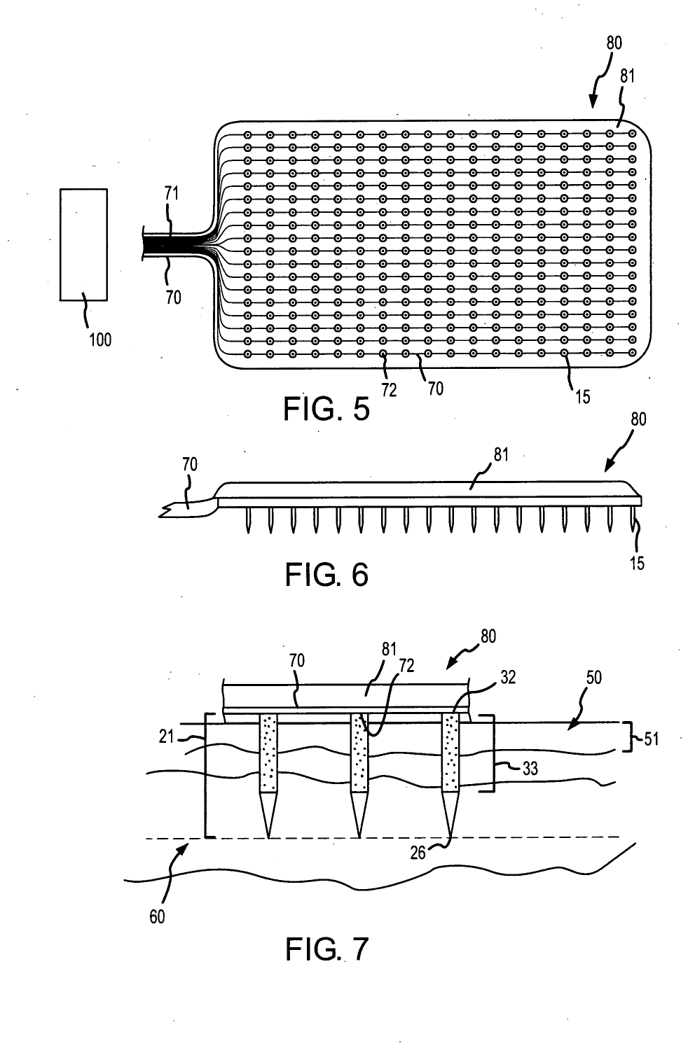

As shown in FIGS. 1-4, in the presently preferred embodiment, the second shaft section 30 is connected to a contact portion 72 of a conductive wire 70. As shown in FIG. 1, the wire 70 is also preferably connected to an adaptor 90 that is connected to an energy source 100 (shown in FIG. 5), such as, for example, all electrical energy source, an ultrasound energy source, or a radiofrequency energy source. The quantity of energy generated by the energy source 100 is preferably regulated by a controller, such as a footplate. Accordingly, an operated can selectively supply a controlled amount of energy, which is conducted to the first shaft section 25 on the needle 15, as the needle 15 is inserted and withdrawn from a patient's skin. An individual needle 15 or a plurality of needles 15 can be inserted perpendicularly or at various angles underneath specific wrinkles or oilier skin conditions of a patient whereby the first shaft section 25 is positioned in the dermal 60 and/or sub-dermal tissue (not shown). A controlled amount of energy can then be directly applied to these tissues, thereby stimulating fibroblast activity, neocollagen production, collagen deposition, collagen contraction, collagen realignment, and subsequent wrinkle reduction and tissue tightening for aesthetic improvement of the patient's skin. Advantageously, while the dermal tissue 60 and/or sub-dermal tissue (not shown) directly receive the energy, the insulating section 31 on the second shaft section 30 prevents or limits the direct application of energy to the superficial epidermal tissue 50 of the patient, thereby protecting this tissue from damage.

Turning now to FIGS. 5-7, a preferred embodiment of a needle system 80 is depicted. As shown, the needle system 80 includes a pad 81 that sheaths at least a portion of a plurality of conductive wires 70. Also shown in FIG. 5, in the preferred embodiment, insulated sections 71 of the wires 70 extends outside the pad 81 whereat they preferably connect to an adaptor 90, which, in turn, is connected to an energy source 100, preferably an adjustable energy source 100, such as, for example, an electrical energy source, an ultrasound energy source, or a radiofrequency energy source. As shown in FIG. 7, the wires 70 sheathed in the electrode pad 81 are exposed, at least in part, and in contact with the contact portions 32 on the needles 15. In the presently preferred embodiment the pad 81 is made of a flexible insulating material, such as, for example, a “type c” electrode material.

Turning now to FIG. 7, the needles 15 are shown partially inserted into openings defined by the pad 81 so that the contact 32 of the needles 15 contact the contacts 72 of the wires 70. In addition to insulating the wires 70, the pad 81 of the presently preferred embodiment of the needle system 80 holds the needles 15 in engagement with the wires 70. Depending on the area of treatment on the patient, the pad 81 may be tailored to include needles 15 of a variety of diameters, angulations, sizes and/or lengths corresponding to specific anatomical areas of by the body. Additionally, the pad 80 may be provided with a variety of anatomical shapes and contours tailored for use on particular anatomical parts of a patient's body.

While this invention has been particularly shown and described with references to various embodiments thereof, it will be understood by those skilled in the art that various changes in form and details may be made therein without departing from the spirit and scope of the invention as defined by the appended claims. For the purpose of teaching preferred principles, some conventional aspects have been simplified or omitted. Those skilled in the art will appreciate variations from these examples that fall within the scope of the invention. Those skilled in the art will appreciate that the features described above can be combined in various ways to form multiple variations of the invention. As a result, the invention is not limited to the specific examples described above, but only by the claims and their equivalents.

1. A needle, comprising:
a) a shaft, including:
i) a pointed end; and
ii) means for directly applying energy to subepidermal tissue in a patient when the needle is inserted into tissue of the patient in order to reduce the appearance of aging signs in tissue.

2. The needle according to claim 1, further comprising an insulating material on a shaft section of the shaft.

3. A needle system, comprising:
a) a wire provided with means for receiving energy; and
b) a needle provided with a shaft and a pointed end, wherein the needle is provided with means for directly applying the energy to subepidermal tissue in a patient when the needle is inserted into tissue of the patient in order to reduce the appearance of aging signs in tissue.

4. (canceled)

5. A needle system, comprising:
a) a wire provided with means for receiving energy;
b) a needle provided with a shaft and a pointed end, wherein the needle is provided with means for directly applying the energy to sub-dermal tissue in a patient when the needle is inserted into tissue of the patient in order to reduce the appearance of aging signs in tissue; and
c) an insulating material on a shaft section of the shaft.

6. (canceled)

7. (canceled)

8. The needle according to claim 5, wherein the shaft section that includes the insulating material is provided with a length that is dimensioned to be greater than or equal to a depth of the epidermal tissue.

9. The needle according to claim 4, wherein the means for directly applying the energy to subepidermal tissue in a patient when the needle is inserted into tissue of the patient in order to reduce the appearance of aging signs in tissue includes a contact portion on the needle that receives energy conducted by the wire and a shaft section that when inserted into the subepidermal tissue, directly applies energy to the subepidermal tissue.

10. The needle according to claim 5, wherein the means for directly applying the energy to subepidermal tissue in a patient when the needle is inserted into tissue of the patient in order to reduce the appearance of aging signs in tissue includes a contact portion on the needle that receives energy conducted by the wire and a shaft section that, when inserted into the subepidermal tissue, directly applies energy to the subepidermal tissue.

11. The needle according to claim 4, further comprising:
a) an energy source, wherein:
i) the wire receives the energy generated by the energy source; and
ii) the means for directly applying the energy to subepidermal tissue in a patient when the needle is inserted into tissue of the patient in order to reduce the appearance of aging signs in tissue includes a contact portion on the needle that receives energy conducted by the wire.

12. The needle according to claim 5, further comprising:
a) an energy source, wherein:
i) the wire receives the energy generated by the energy source; and
ii) the means for directly applying the energy to subepidermal tissue in a patient when the needle is inserted into tissue of the patient in order to reduce the appearance of aging signs in tissue includes a contact portion on the needle that receives energy conducted by the wire.

13. A needle system, comprising:
a) a pad that sheaths a plurality of conductive wires;
b) a plurality of needles that include elongated shafts provided with pointed ends, first shaft sections, and second shaft sections, wherein the first shaft sections and the second shaft sections include a conductive material and the second shaft sections include insulated shaft sections and contact portions, wherein:
i) the contact portions are exposed to receive energy;
ii) the second shaft sections are adapted to conduct the energy to the first shaft sections;
iii) the insulated shaft sections are provided with lengths dimensioned according to a depth of epidermal tissue; and
c) the plurality of conductive wires are adapted to conduct the energy.

14. The needle system according to claim 13, wherein the plurality of conductive wires conduct the energy to the contact portions on the second shaft sections.

15. The needle system according to claim 13, wherein the lengths are dimensioned to be greater than or equal to the depth of epidermal tissue.

16. The needle system according to claim 13, further comprising an adaptor that is configured to connect the conductive wires to an energy source.

17. The needle system according to claim 13, wherein the shaft of at least one needle is angulated.

18. The needle system according to claim 13, wherein the plurality of needles include at least two needles, wherein the shafts of the two needles are provided with different diameters.

19. The needle system according to claim 13, wherein the plurality of needles include at least two needles, wherein the shafts of the two needles are provided with different shaft lengths.

20. The needle system according to claim 13, wherein the plurality of needles include at least two needles, wherein the shafts of the two needles are provided with different angulations.

21. The needle system according to claim 13, wherein the pad holds the needles in contact with the wires.

22. The needle system according to claim 13, wherein the pad includes an insulating material.

23. A needle system, comprising:
a) an energy source;
b) a pad that sheaths a plurality of conductive wires that conduct energy generated by the energy source;
c) a plurality of needles that include elongated shafts provided with pointed ends, first shaft sections, and second shaft sections, wherein the first shaft sections and the second shaft sections include a conductive material and the second shaft sections include insulated shaft sections and contact portions, wherein:
i) the contact portions on the second shaft sections receive energy generated by the energy source;
ii) the second shaft sections conduct the energy to the first shaft sections;
iii) the insulated shaft sections are-provided with lengths dimensioned according to a depth of epidermal tissue; and
d) the plurality of conductive wires conduct the energy generated by the energy source.

24. The needle system according to claim 23, wherein the plurality of conductive wires conduct the energy to the contact portions on the second shaft sections.

25. The needle system according to claim 23, wherein the lengths are dimensioned to be greater than or equal to the depth of epidermal tissue.

26. The needle system according to claim 23, further comprising an adaptor that connects the conductive wires to the energy source.

27. The needle system according to claim 23, further comprising a controller for adjusting the output of energy from the energy source.

28. The needle system according to claim 23, further comprising a footplate for adjusting the output of energy from the energy source.

29. The needle system according to claim 23, wherein the shaft of at least one needle is angulated.

30. The needle system according to claim 23, wherein the plurality of needles include at least two needles, wherein the shafts of the two needles are provided with different diameters.

31. The needle system according to claim 23, wherein the plurality of needles include at least two needles, wherein the shafts of the two needles are provided with different shaft lengths.

32. The needle system according to claim 23, wherein the plurality of needles include at least two needles, wherein the shafts of the two needles are provided with different angulations.

33. The needle system according to claim 23, wherein the energy source is an electrical energy source.

34. The needle system according to claim 23, wherein the energy source is an ultrasound energy source.

35. The needle system according to claim 23, wherein the energy source is a radio frequency energy source.

36. The needle system according to claim 23, wherein the pad holds the needles in contact with the wires.

37. The needle system according to claim 23, wherein the pad includes an insulating material.

38. The needle according to claim 1, wherein the means for directly applying the energy to subepidermal tissue in a patient when the needle is inserted into tissue of the patient in order to reduce the appearance of aging signs in tissue includes a contact portion on the needle that receives energy conducted by the wire.

39. The needle according to claim 1, further comprising:
a) an energy source, wherein the means for directly applying the energy to subepidermal tissue in a patient when the needle is inserted into tissue of the patient in order to reduce the appearance of aging signs in tissue includes a contact portion on the needle that receives energy generated by the energy source.

40. The needle according to claim 1, further comprising:
a) an energy source and a wire that conducts energy generated by the energy source, wherein:
i) the means for directly applying the energy to subepidermal tissue in a patient when the needle is inserted into tissue of the patient in order to reduce the appearance of aging signs in tissue includes:
(1) a contact portion on the needle that receives the energy conducted by the wire;
(2) and a shaft section that, when inserted into the subepidermal tissue, directly applies energy to the subepidermal tissue; and
b) an insulating material on another shaft section.
